(19) United States
(12) Patent Application Publication
(10) Pub. No.: US 2025/0090350 A1
(43) Pub. Date: Mar. 20, 2025

(54) Wirelessly Actuated Ciliary Stent

(71) Applicant: VANDERBILT UNIVERSITY, Nashville, TN (US)

(72) Inventors: Xiaoguang Dong, Nashville, TN (US); Yusheng Wang, Nashville, TN (US)

(21) Appl. No.: 18/882,829

(22) Filed: Sep. 12, 2024

Related U.S. Application Data
(60) Provisional application No. 63/616,945, filed on Jan. 2, 2024.
(60) Provisional application No. 63/582,872, filed on Sep. 15, 2023.

(51) Int. Cl. A61F 2/48; A61F 2/00; A61F 2/04

(52) U.S. Cl. CPC A61F 2/48; A61F 2/0077; A61F 2002/043; A61F 2002/046; A61F 2210/0076; A61F 2210/009; A61F 2240/001

(57) ABSTRACT

Disclosed are various approaches for an apparatus having a tubular member which has a radially external side and a radially internal side. The apparatus can further include a plurality of artificial cilia disposed circumferentially on the radially internal side of the tubular member, where the plurality of artificial cilia forming arrays extending longitudinally along the radially internal side of the tubular member.

### CROSS-REFERENCE TO RELATED APPLICATIONS

This application claims priority to, and the benefit of U.S. Provisional Patent Application 63/582,872 entitled “WIRELESSLY ACTUATED CILIARY AIRWAY STENT FOR EXCESSIVE MUCUS REMOVAL” filed on Sep. 15, 2023, and U.S. Provisional Patent Application 63/616,945 entitled “WIRELESSLY ACTUATED CILIARY AIRWAY STENT FOR EXCESSIVE MUCUS REMOVAL” filed on Jan. 2, 2024, which are incorporated by reference as if set forth herein in their entirety.

### BACKGROUND

Cilia are slender, hairlike projections which can be found on numerous different cell bodies. In mammals, these cilia are often motile, or capable of motion, and move in coordinated metachronal waves. The motile cilia and their metachronal waves are often necessary for moving various substances through the body. For example, cilia in respiratory tissues move mucus through the respiratory organs.

### SUMMARY

In accordance with the purpose(s) of this disclosure, as embodied and broadly described herein, the disclosure, in various aspects, relates to artificial ciliary stents and methods of use thereof.

Aspects of the present disclosure provide for the design, integration, and control of artificial cilia in stents for placement in human patients. Embodiments of the present disclosure include: a tubular member, a plurality of magnetic cilia, and a magnetic actuation system. The tubular member can have a radially external side and a radially internal side. The plurality of magnetic cilia can be disposed circumferentially on the radially internal side of the tubular member and extend along the radially internal side of the tubular member from an entry end to an exit end. The magnetic actuation system can be configured to generate metachronal wavelike motion of the plurality of magnetic cilia. In some embodiments, the plurality of magnetic cilia further comprises a first plurality of magnetic cilia disposed on a first semi-cylinder portion of the tubular member, and a second plurality of magnetic cilia disposed on a second semi-cylinder portion of the tubular member. The first plurality of magnetic cilia can have a first magnetization profile, and the second plurality of magnetic cilia can have a second magnetization profile. The first magnetization profile and the second magnetization profile are configured such that the magnetic actuation system can cause both the first plurality of magnetic cilia and the second plurality of magnetic cilia to produce a net flow from the entry end to the exit end of the tubular member. In addition, the system can further comprise a hydrogel coating on each of the plurality of magnetic cilia. In some embodiments, the magnetic actuation system comprises a rotating Halbach array of a plurality of magnets. Individual magnetic cilia of the plurality of magnetic cilia can further comprise a first layer of microstructured polydimethylsiloxane (PDMS); a magnetic composite layer disposed atop the first layer of PDMS, where the magnetic composite layer has a programmed magnetization profile to achieve metachronal wave-like deformation when actuated by an external magnetic field; and a second layer of microstructured PDMS disposed atop the magnetic composite layer. Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an illustration of the concept and design of a ciliary airway stent (CAS). FIG. 1(A) is an illustration of the concept of a ciliary airway stent implanted inside a human trachea for pumping out excessive mucus. FIG. 1(B) is an illustration of the proposed airway stent integrated with magnetic cilia and hydrogel coating. FIG. 1(C) is an illustration of artificial cilia with bio-inspired metachronal waves. An antiplectic metachronal wave is encoded in the cilia arrays, where the direction of the fluid flow is opposite to that of the wave prorogation. FIG. 1(D) is an illustration of the non-reciprocal motion of a single artificial cilium. The swiping area of a single artificial cilium is marked with a gray-shaded area. Top (red) arrow: power stroke direction. Bottom (blue) arrow: recovery stroke direction. FIG. 1(E) is an illustration of mucus transportation by a cilia array with hydrophilic or hydrophobic surface coating. FIG. 1(F) is two images of the CAS being compressed and after releasing the compression to show the resilience of the cilia structure. FIG. 1(G) is two images of the CAS transporting porcine mucus inside a trachea phantom. In all figures, the scale bars represent 5 mm.

FIG. 2 is an illustration of a fabrication process of a ciliary airway stent. FIG. 2(A) is an illustration of the multi-step fabrication process of a magnetic cilia patch combining molding and laser cutting. FIG. 2(B) is an optical microscope image of the cross section of a magnetic cilium with PDMS micro-pillar structures for encapsulation and robust hydrogel coating. FIG. 2(C) is an illustration of the example process of programming designed magnetization profiles of the artificial cilia arrays for non-reciprocal motion and metachronal waves. FIG. 2(D) is an illustration of the process of coating hydrogel on a cilia patch with PDMS micro-pillar structures. FIG. 2(E) is two optical images of the fabricated cilia patch without (top image) and with (bottom image) a magnetic field applied, respectively. FIG. 2(F) is an illustration of the process of assembling magnetic cilia patches on a silicone airway stent.

FIG. 3 shows a characterization and optimization of the pumping performance of a cilia array in viscous fluids. FIG. 3(A) is a schematic of the experiment setup for actuating a magnetic cilia array. A cilia array is submerged in a viscous fluid and actuated by a rotational magnetic field in the x-y plane. The key design parameters of a cilia array include the thickness: t, width: w, length: L, and spacing: do. FIG. 3(B) is a graph of a simulated magnetic field magnitude distribution on the x-y plane at z=2.1 cm. The cilia array location is marked in a red-dotted rectangle with its center marked with a red dot. FIG. 3(C) is a graph of the measured magnetic field (at x=0, μ=0, z=2.1 cm) as a function of time. FIG. 3(D) shows video frames of the motions of two types of cilia used in the stent and illustration of their tip trajectories. A rotating magnetic field with a magnitude B=40 mT and a frequency f=1 Hz (T=1 s) in the counterclockwise direction in the x-y plane is applied. The viscous liquid used is syrup (Dynamic viscosity μ=5000 mPa·s). FIGS. 3(E-F) show the swiping area ratio rates (SARR) of Type I (E) and Type II (F) cilia as function of the magnetic field frequency f when B=40 mT. SARR=SA·f/(d″ ·d!). FIG. 3(G) shows the average particle transportation speeds (ūₚ), for two types of cilia arrays as a function of β. β is the angle difference between the cilia body plane (x-y plane) and the magnetic field plane. ūₚ is defined as the average speed of N=5 particles traveling through a rectangular-shaped area of 3d_{!}×L (in x, y). FIG. 3(H) shows the SARRs of two types of cilia in liquids of different dynamic viscosities. B=40 mT, f=1 Hz.

FIG. 4 is an investigation of the effect of surface coating on the performance of pumping viscous fluids. FIG. 4(A) is three optical images of cilia surface (i) without or (ii) with the PDMS layer, and (iii) confocal fluorescent images of the hydrogel layer mixed with fluorescence dye (Rhodamine 6G). In (iii), from the top to the bottom, three pictures are the cross section of the hydrogel layer, hydrogel on the pillars, and hydrogel on the surface. Scale bars, 50 μm. FIG. 4(B) shows images of the static water contact angles for PDMS coated cilia surfaces with or without hydrogel coating. FIG. 4(C) shows SARRs of the cilia arrays with or without hydrogel coating. The labels “I” and “II” represent the cilia magnetization types. FIG. 4(D-E) shows video frames of the wetting-based transportation for cilia arrays with or without hydrogel coating in (D) syrup (0.3 mL, μ=5000 mPa·s), and (E) mucus (0.2 mL, μ=11300 mPa·s). The magnetic fields are all in the x-y plane with B=40 mT, f=1 Hz.

FIG. 5 is a characterization of the pumping performance of the ciliary airway stent. FIG. 5(A) shows the experimental setup for actuating and characterizing the ciliary stent. The system includes a silicone trachea phantom, a customized peristaltic pump for feeding liquid into the phantom, a sample holder with an adjustable tilting angle (a), a rotating magnet, and an endoscope. The distance between the central axis of the stent and the rotation axis of the magnet is denoted as D. FIG. 5(B) is an illustration of the liquid transportation inside the CAS. The average transportation speed is given by u=s/Δt, where s is the distance between one end of the stent to the farthest cilium and Δt is the transportation time. FIG. 5(C) shows video frames of five different types of airway stents at t=100 seconds. From left to right: Stent I—stent with hydrogel coated cilia under magnetic actuation; Stent II—stent with actuated cilia but not hydrogel coating; Stent III —stent with hydrogel coated cilia but with no magnetic actuation; Stent IV—stent with neither hydrogel coating nor magnetic actuation; Stent V—stent with neither cilia nor hydrogel coating. FIG. 5(D) shows the average transportation speed of the five types of airway stents. Dynamic viscosity of the syrup: μ=5000 mPa·s. FIG. 5(E) shows the average transportation speed of Stent I as a function of liquid viscosities. FIG. 5(F) shows the average transportation speed of Stent I with different tilting angles α. μ=5000 mPa·s. FIG. 5(G) shows the average transportation speed of Stent I with different rotating angles γ, which are the angles between the division line and the rotating axis of the magnet. μ=5000 mPa·s. For all experiments, B=40 mT, f=2 Hz.

FIG. 6 is a demonstration of transporting mucus in a lung phantom and visualization by medical imaging. FIG. 6(A) shows video frames of five different types of stents pumping in mucus at t=46 seconds. FIG. 6(B) shows transportation rates of five different types of stents in mucus with μ=8705 mPa·s. FIG. 6(C) shows video frames of the optimal airway stent (Stent I) pumping mucus with μ=8705 mPa·s at different time steps. FIG. 6(D) shows the average mucus transportation speed of Stent I as a function of mucus viscosities. FIG. 6(E) shows an isometric of mucus transportation in a half stent. FIG. 6(F) is a series X-ray images of Stent I at different time steps to visualize metachronal waves. Cilia with motion are highlighted with red dotted frames. Markers are highlighted with a yellow dotted ellipse. In all figures, scale bars represent 5 mm.

FIG. 7.1 shows a characterization of the PDMS coating. The PDMS is mixed with Rhodamine 6G and visualized by confocal fluorescent imaging (LSM 710, Carl Zeiss Technology).

FIG. 7.2 is an illustration of artificial cilia distribution inside the stent with respect to types. Two types of the artificial cilia are distributed oppose to each other so that they can generate flows with the same direction under one magnetic field.

FIG. 7.3 shows an experimental setup for testing artificial cilia patches with a Halbach array. The magnetic field at the artificial cilia array is 40 mT.

FIG. 7.4 shows dynamic spreading of liquids of different viscosities. FIG. 7.4(A) is video frames of the spreading behaviors of liquids in different viscosities when the artificial cilia are actuated by external magnetic fields. FIG. 7.4(B) is average spreading velocities ū_{f} of liquids in different viscosities when artificial cilia are actuated by external magnetic fields. ū_{f}=lf/Δt, where if is the traveled distance of the liquid front and Δt=24 s. FIG. 7.4(C) is video frames of the spreading behaviors of liquids in different viscosities when the artificial cilia are not being actuated. FIG. 7.4(D) shows average spreading velocities of liquids in different viscosities when the artificial cilia are not being actuated. The magnetic fields are in the x-y plane with B=40 mT and f=2 Hz. In all experiments, liquids: syrup and water mixture, volume: 0.25 mL.

FIG. 7.5 shows dynamic spreading of liquids by rod-shaped or beam-shaped artificial cilia. FIG. 7.5(A) shows video frames of syrup spread by rod-shaped artificial cilia. FIG. 7.5(B) shows video frames of syrup spread by beam-shaped artificial cilia. FIG. 7.5(C) shows video frames of fluid-structure interaction between syrup and rod-shaped artificial cilia. FIG. 7.5(D) shows video frames of fluid-structure interaction between syrup and beam-shaped artificial cilia. The magnetic fields are in the x-y plane with B=40 mT and f=2 Hz. In all experiments, liquid: syrup, volume: 0.25 mL, μ=6600 mPa·s.

FIG. 7.6 shows a graph of the shear-thinning rheological property of mucus. The mucin-water mixture with a ratio of 1:6.

FIG. 7.7 shows an experimental setup for testing the stent. A peristaltic pump with a valve is used to feed the liquid with a constant speed. The stent is placed inside the phantom and actuated by the magnet. An endoscope is implemented for imaging.

FIG. 7.8 shows the magnitude of the magnetic field as a function of the actuation distance. The distance D is the centerline of the stent to a spherical permanent magnet (diameter: 32 mm, NdFeB, N42).

FIG. 7.9 is an illustration of the stent orientations and rotational directions of the magnet. FIG. 7.9(A) shows that stent orientations (γ) are defined as the angles between the division line (1) and the rotational axis of the magnet. FIG. 7.9(B) shows the rotational direction of the magnet about x axis, with respect to the stent orientations, assuming the stent is pumping in +y direction.

FIG. 7.10 is an illustration of markers for tracking stent orientations in X-ray imaging. Triangular markers made of metal composites are attached to one end of the stent.

FIG. 7.11 is a characterization of the dynamic spreading of liquid by artificial cilia arrays of different spacings. FIG. 7.11(A) is video frames of syrup transportation with artificial cilia of different spacings. Spacing ratio=dc/L, where dc is the spacing of artificial cilia and L is the length of one artificial cilium. FIG. 7.11(B) shows the average spreading velocities of syrup by artificial cilia arrays of different spacing. The magnetic fields are all in the x-y plane with B=40 mT and f=2 Hz. In all experiments, liquid: syrup, volume: 0.25 mL, μ=6600 mPa·s.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, biomedical engineering and mechanical engineering techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### DISCUSSION

Disclosed are various approaches for the design, integration, and control of arrays of artificial cilia on 3-dimensional (3D) surfaces. While the Example herein is focused on the design, integration, and control of artificial cilia in airway stents, the principles of the present disclosure can also be extended to various other medical devices, 3D structures, or use cases as can be appreciated. The approaches disclosed herein provide a new means for moving viscous liquids or other debris by means of artificial cilia. In many applications, small-scale cilia-like devices can be useful to manipulate fluids in narrow and confined spaces. Particularly, in the example of airway stents, the use of cilia-like structures can help prevent blockages of the stents and reduce the risk of related infections.

Current airway stent technologies include the use of silicone stents and metal stents. Metal stents often result in ingrowth into the soft tissues surrounding the stent and require complex removal procedures. Silicone stents, contrarily, are easy to remove and have a lower risk of ingrowth. However, silicone stents impair the function of the epithelial cilia in a patient's airway.

Accordingly, various embodiments of the present disclosure are directed to systems and methods for using a magnetically actuated cilia-like structures in an airway stent to clear mucus. To accomplish this, a system can be arranged having a plurality of magnetically actuated cilia-like structures disposed within a tubular member serving as the airway stent. Additionally, the system can include a magnetic actuator capable of producing a magnetic field which actuates the cilia-like structures.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

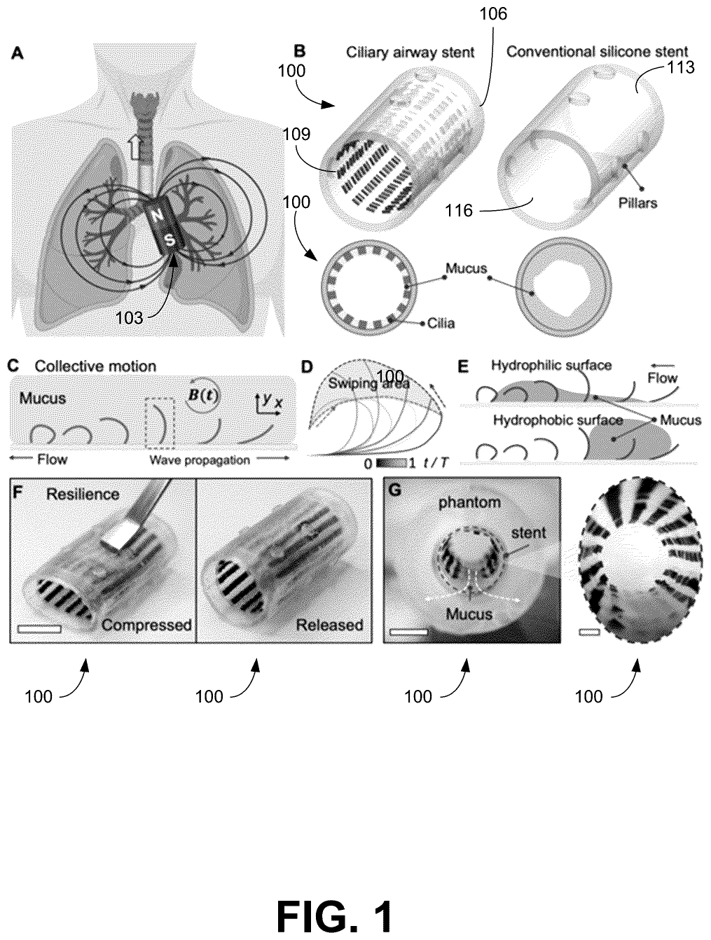

With reference to FIG. 1, shown is the concept and design of a ciliary airway stent (CAS) 100. FIG. 1(A) shows the placement of a CAS 100 in a patient with a representation of a magnetic actuator 103 generating a magnetic field to operate the CAS 100. The CAS 100 can be representative of any ciliary airway stent 100 having a hollow tubular member 106 and a plurality of artificial cilia 109. The hollow tubular member 106 can be comprised of silicone, natural rubbers, polyurethanes, polymers, or other flexible, nonmagnetic, and biocompatible material. The hollow tubular member 106 have an internal diameter of about 4-16 mm, an external diameter of about 6-18 mm, and a length of about 10-80 mm. The hollow tubular member 106 can have a radially external side 113, which is the external surface of the hollow tubular member 106 which will be in contact with a patient's airway, and a radially internal side 116 which is the internal surface of the hollow tubular member 106 through which air and potentially other fluids will flow. In some embodiments, the hollow tubular member 106 can be manufactured through a sacrificial molding method. For example, the hollow tubular member 106 can be fabricated by using a negative mold with hollow structures. The negative mold can be 3D printed with water-soluble Polyvinyl alcohol (PVA) using a Fused Deposition Modeling (FDM) 3D printer. Liquid Polydimethylsiloxane (PDMS) can be injected into the negative mold, and subsequently cured on a hotplate at a temperature of approximately 40° C. for about 48 hours. After the PDMS is cured inside the mold, the whole structure can be placed in hot water (−70° C.) and the mold dissolved to obtain the hollow tubular member 106. However, the hollow tubular member 106 can be fabricated according to various other methods as well.

The plurality of artificial cilia 109 can be disposed within the hollow tubular member 106 along the radially internal side 116. Each of the individual artificial cilia 109 can be formed in a bar-like, or rectangular prism shape, having dimensions of about 1.75 mm×1.5 mm×0.07 mm. In some embodiments, the artificial cilia 109 are formed in a cylindrical shape, or other oblong 3D shape. As shown in FIG. 1(B), the artificial cilia 109 can be aligned in arrays within the hollow tubular member 106 such that when they are not actuated, the artificial cilia 109 do not obstruct the flow of fluids. In some embodiments, the arrays of artificial cilia 109 are disposed circumferentially about the radially internal side 116 of the hollow tubular member 106, as shown in FIG. 1(B, F, G). In some embodiments, the arrays of artificial cilia 109 are disposed longitudinally through the hollow tubular member 106.

The artificial cilia 109 can be actuated by subjecting the artificial cilia 109 to a rotating magnetic field. In some embodiments, the artificial cilia 109 are encoded to produce metachronal waves as shown in FIG. 1(C). Metachronal waves can be encoded by programming an offset angle in the neighboring cilia magnetization profiles. This offset angle can result in a time lag in the rotational movement of downstream artificial cilia 109 and cause a traveling wave to be formed. The artificial cilia 109 can be encoded to generate an antiplectic or a symplectic wave. In some embodiments, the artificial cilia 109 can be encoded to produce non-reciprocal motion which consists of power strokes and recovery strokes, as shown in FIG. 1(D).

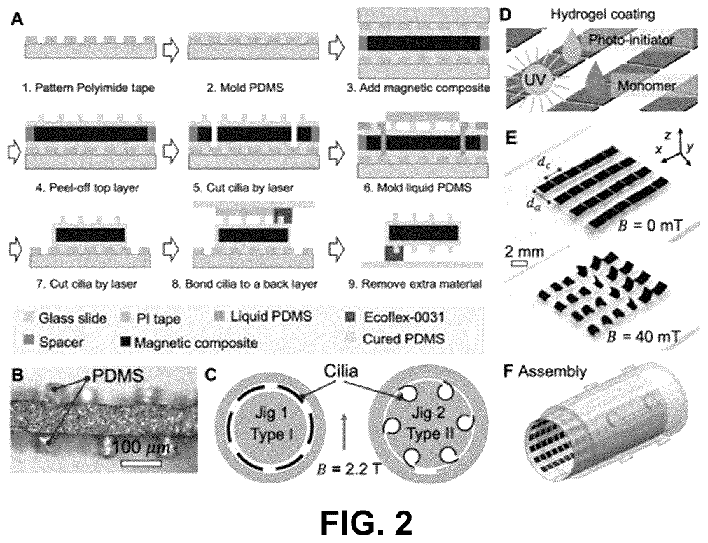

Moving on to FIG. 2, shown is an example fabrication process for the artificial cilia 109. The artificial cilia 109 can be comprised of a multi-layer composite material. In some embodiments, the artificial cilia 109 have a magnetic core encapsulated in PDMS. The artificial cilia 109 can be manufactured by way of a method of laser machining and micro-molding. For example, the artificial cilia 109 can be formed from a three-layer structure including a first layer of microstructured PDMS, a magnetic composite layer, and a second layer of microstructured PDMS. The microstructures on the PDMS can result from a molding process wherein liquid PDMS is poured over a Polyimide (PI) tape having an array of holes and cured. Next, a layer of magnetic composite can be used to form the magnetic core of the artificial cilia 109. In some embodiments, the magnetic composite material can be composed of Neodymium Iron Boron magnetic particles (NdFeB), Samarium Cobalt (SmCo), Alnico, Ferrite, or other magnetic material, and silicone rubber. The magnetic composite can have a programmable magnetization profile which can be programmed to achieve the desired deformation of the artificial cilia 109 when actuated by the magnetic actuator 103. The magnetic composite layer can be cured between two layers of PDMS to form a three-layer structure. Next, the artificial cilia 109 can be cut from the three-layer structure. In some embodiments, the artificial cilia 109 can be cut using a laser cutter. In some embodiments, the artificial cilia 109 can be cut based at least in part on the pattern, placement, structure, or other feature of the microstructures on the outer PDMS layers. If, during laser cutting, any of the magnetic composite core is exposed, more liquid PDMS can be applied and cured to cover the magnetic composite. After curing, the coated artificial cilia 109 can be bonded to a thin layer of PDMS to form a patch which can then be bonded to a radially internal side 116 of a hollow tubular member 106. In some embodiments, there can be 36 artificial cilia 109 per patch of 24×7.5 mm dimensions.

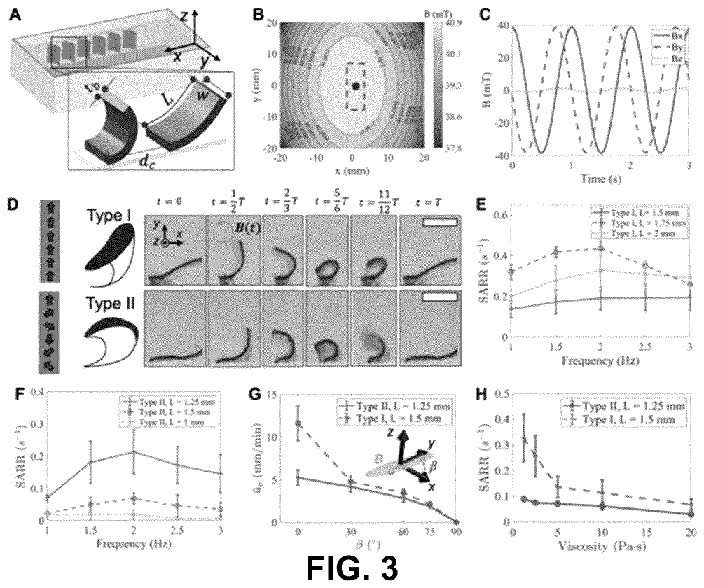

Turning now to FIG. 3, shown are various representations of the characterization and optimization of the pumping performance of the artificial cilia 109. The magnetic actuator 103 can be representative of any device, system, or component, which is capable of producing a rotating magnetic field. In some embodiments, the magnetic field can represent that shown in FIG. 3(B). The magnetic actuator 103 can be a Halbach array made of a number of magnets. In some embodiments, the Halbach array includes about 3-15 magnets which can be rotated using a motor or other mechanism. The magnetic actuator 103 can be a motorized Halbach array as described, although in other embodiments, a different magnetic actuator 103 can be used. For example, a single magnet rotated by a motor can be used as a magnetic actuator 103 to generate a rotating magnetic field having a controlled frequency and magnitude.

According to various examples, two or more kinds of artificial cilia 109 can be included in the hollow tubular member 106 in order to produce a uniform flow. As shown in FIG. 2(C) and FIG. 3(D), at least two types of artificial cilia 109 can be included on opposite sides of the hollow tubular member 106 such that a single magnetic actuator 103, and in turn, a single rotating magnetic field, could act upon the two different types of artificial cilia 109 from different angles and produce the same waveform. For example, as shown in FIG. 3(D), the two types of artificial cilia 109 can be programmed to have differing magnetization profiles which are configured such that when a single rotating magnetic field is applied, a net flow in the same direction is produced by each type of artificial cilia 109. In some embodiments, a first type of artificial cilia 109 is integrated into a first semi-cylinder portion of the hollow tubular member 106 and a second type of artificial cilia 109 is integrated into a second semi-cylinder portion of the hollow tubular member 106.

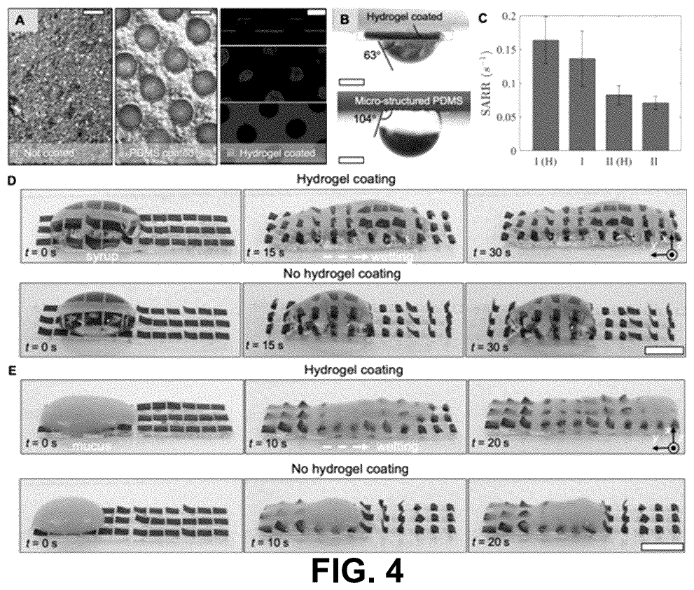

Moving on to FIG. 4, shown is an investigation of the effect of surface coating on the performance of pumping viscous fluids. The CAS 100 can further include a hydrogel coating. In some embodiments, the hydrogel coating is incorporated on the radially internal side 116 of the hollow tubular member 106. In some embodiments, the hydrogel coating is disposed on the artificial cilia 109. Use of a hydrogel can operate to spread fluids over a greater surface area within the hollow tubular member 106 and can cause improved results for transportation of those fluids. Coating the CAS 100 can include the steps of first submerging the artificial cilia 109 in a benzophenone-ethanol mixture, drying the artificial cilia 109, applying polyethylene glycol diacrylate (PEGDA) to the artificial cilia 109, and then curing the PEGDA with ultraviolet (UV) light. In some embodiments, other forms of hydrogel coatings and hydrogel coating methods can be used on the CAS 100 to enhance fluid transportation.

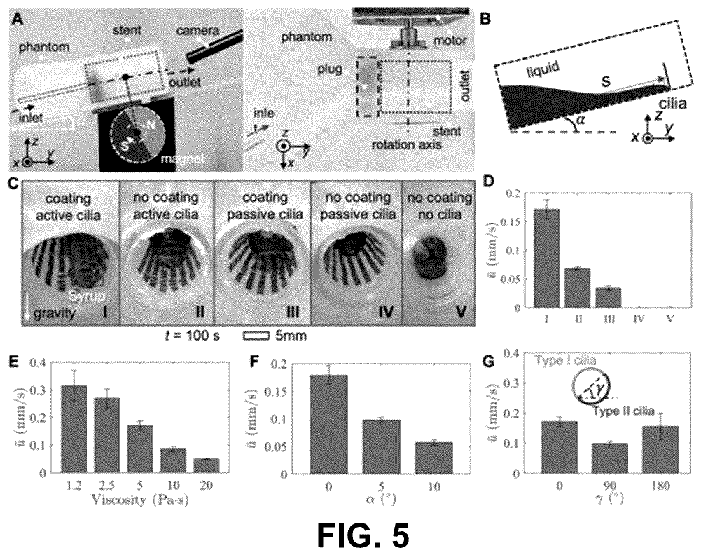

Next, at FIG. 5, shown is a characterization of the pumping performance of the CAS 100. The patches of artificial cilia 109, as described in the discussion of FIG. 2, can be integrated into a hollow tubular member 106 to form the CAS 100. As described in the discussion of FIG. 3, two or more types of artificial cilia 109 can be integrated into a hollow tubular member 106 to ensure even wave production from the artificial cilia 109 when activated. In some embodiments, a first type of artificial cilia 109 are integrated into a first semi-cylinder half of the hollow tubular member 106 and a second type of artificial cilia 109 are integrated into a second semi-cylinder half of the hollow tubular member 106. In such embodiments, the angle (γ) between the division line (between the first semi-cylinder half and the second semi-cylinder half) and the rotation axis of the magnetic field can vary between 0° and 180°. As shown in FIG. 5(G), an angle of μ=90⁰ is less effective than angles closer to 0° or 180°. Accordingly, the position of the magnetic actuator 103 relative to the orientation of the CAS 100 can be optimized to produce a greater flow speed. Similarly, the orientation of the CAS 100 when implanted in a patient can be optimized for improving flow as well.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

### Example A

### Introduction

Cilia are tiny slender structures that widely exist in various biological systems with critical functions such as pumping and mixing fluids, propulsion for mobility, and sensing environmental fluid flows. Inspired by the active fluid transportation function of biological cilia such as the epithelial cilia in the lung, small-scale cilia-like devices that can manipulate fluids in narrow and confined spaces have great potential in microfluidics, biomechanics, biomedical engineering, and other applications. Despite recent advances in versatile artificial cilia for fluid manipulation, prior studies mostly focused on developing artificial cilia for pumping water and other synthetic fluids for lab-on-a-chip microfluidic applications. So far, the design and control of artificial cilia for transporting viscous mucus in confined and tubular structures remains challenging and medical devices such as airway stents with ciliary function are still missing. On the other hand, airway stents play an important role in various lung diseases such as lung cancer, Cystic fibrosis (CF), and Chronic obstructive pulmonary disease (COPD). They are hollow and flexible tubes made of metal or silicone which can provide radial support in the trachea or bronchi of a patient when there is airway narrowing or stricture. Both silicone stents and metal stents have been reported with several well-known limitations. First, silicone stents are usually preferred as they are easy to remove and have a relatively low cost. However, they frequently become occluded with a mixture of saliva and mucus, due to the loss in the ciliary function which is responsible for pumping viscous liquids upward. This may result in serious airway obstruction and chronic infection. Invasive mucus clearance intervention via bronchoscopy is frequently needed which increases morbidity, healthcare costs, and even mortality. In contrast, self-expandable metal stents do not impair the ciliary function, are relatively easier to be deployed and to be captured under X-ray imaging, but the ingrowth into soft tissues makes stent removal difficult and may further require complex and dangerous debridement and extraction procedures. Other complications include airway bleeding, infection, granulation tissue hyperplasia, and subcutaneous emphysema, among others. To prevent tissue ingrowth, the metal stent needs be covered with a layer of silicone. However, like the silicone stents, this meshed metal stents will also impair the ciliary function on the endoluminal surface of the trachea. Thus, creating airway stents which do not impair the function of transporting mucus, while avoiding tissue ingrowth, remains challenging but an urgent clinical need.

Integrating artificial cilia on silicone or meshed metal airway stents could be a potential solution to address the challenge. Artificial cilia have been explored in prior works, which mimic the physiological function of biological ciliary structures by emulating the collective motion and single cilium motion for pumping fluids at a low Reynolds number. Existing artificial cilia have been shown to be actuated by pneumatic, electric, light, as well as external magnetic fields and other stimuli. Among them, magnetic cilia have drawn attention due to the complex shapes and motions that they can achieve. However, prior studies mostly focused on developing artificial cilia for lab-on-a-chip microfluidic applications, which have not been shown in implantable medical devices yet due to the lack of a systematic method of design and integration of the magnetic cilia with medical devices and control their behaviors.

Here a method is proposed to design, integrate and control magnetic artificial cilia inside airway stents, which allows efficient removal of excessive mucus. First, novel magnetic artificial cilia arrays are designed by encoding the non-reciprocal motion and metachronal waves inspired by biological cilia for transporting mucus efficiently. The stent's geometric and material parameters, as well as the external magnetic fields, are designed to maximize the mucus transportation speeds. Second, a hydrogel coating on the artificial cilia is also proposed as a lubricant layer inspired by the periciliary layer in airway cilia for enhanced mucus transportation. A systematic quantification of the performance of the proposed ciliary airway stent is presented and compared to those stents without artificial cilia or hydrogel coating. Lastly, a novel ciliary airway stent that can transport viscous porcine mucus in a lung trachea phantom even faster than the respiratory cilia in a healthy human lung is demonstrated. The proposed method could potentially overcome the limitation of covered and silicone airway stents, while avoiding the risks associated with tissue ingrowth of conventional metal airway stents. The proposed method of integrating artificial cilia on medical stents also has the penitential to be used for creating versatile ciliary stents for pumping body fluids in the human body in a minimally invasive manner. It thus paves the way towards effective treatment of various diseases with cilia impairment and improve the quality of life.

### Results

### Working Principle, Design, and Fabrication of the Ciliary Airway Stent

As a proof-of-concept, a silicone airway stent with integrated artificial cilia is designed, but the proposed method is generic to other airway stents with covered meshes to prevent tissue ingrowth. As shown in FIG. 1(A), the ciliary airway stent (CAS) is designed to be implanted in the trachea of a human lung to pump out the excessive mucus due to the impairment of biological cilia, in addition to providing radial support to the trachea or main bronchi with strictures. A rotating magnetic field generated by a portable magnetic actuation system is used to actuate the CAS remotely. FIG. 1(B) shows the components of a CAS where artificial cilia made of magnetic composite material in a beam shape are mounted on the stent inner surface. The artificial cilia are aligned along the inner surface of the stent when not actuated to minimize the obstruction of the air flow. The CAS is hypothesized to pump out the excessive mucus to increase the cross section area of the airway compared with a pure silicone stent.

The design process involves both the artificial cilia and the stent. First, as one symmetry-breaking mechanism, FIG. 1(C) shows that the artificial cilia in an array along the stent body axis are encoded with a metachronal wave by programming an offset angle in the neighboring cilia magnetization profiles. Subject to a rotational magnetic field, a traveling wave formed by the neighboring cilia is realized due to the time lags in a rotational buckling motion. Based on whether the net fluid flow and the wave propagation are in the same direction or not, metachronal waves are classified as symplectic or antiplectic wave, respectively. Particularly, a specific antiplectic wave is encoded inside all cilia arrays, as it has been shown to allow more efficient fluid pumping at a low Reynolds number (Re). Second, as another core mechanism for viscous fluid pumping, FIG. 1(D) and movie S1 shows that the cilium has a programmed non-reciprocal motion which consists of power and recovery strokes when the cilium tip follows different trajectories. The non-reciprocal motion indicates that the trajectory of a cilium tip r^{tip}(t) (t: time) forms a net area within a beating cycle T. The net area is defined as Swiping Area (SA) given by SA=∫₀^{T}r_{y}^{tip} dx(t), which is widely employed as a metric for the cilium's pumping performance in a low-Re environment. By programming the magnetization profile of a cilium, a positive or negative SA can be realized allowing different fluid transportation direction subject to the same rotating magnetic field. Third, a hydrophilic surface property was discovered to improve the CAS's pumping performance by a wetting effect, as illustrated in FIG. 1(E). Lastly, FIG. 1(F,G) shows a prototype of the CAS. FIG. 1(F) shows that the stent is elastic and could resist to forces applied on the stent. FIG. 1(G) shows that in an endoscope view the stent inside a trachea phantom could pump porcine mucus from one side to the other.

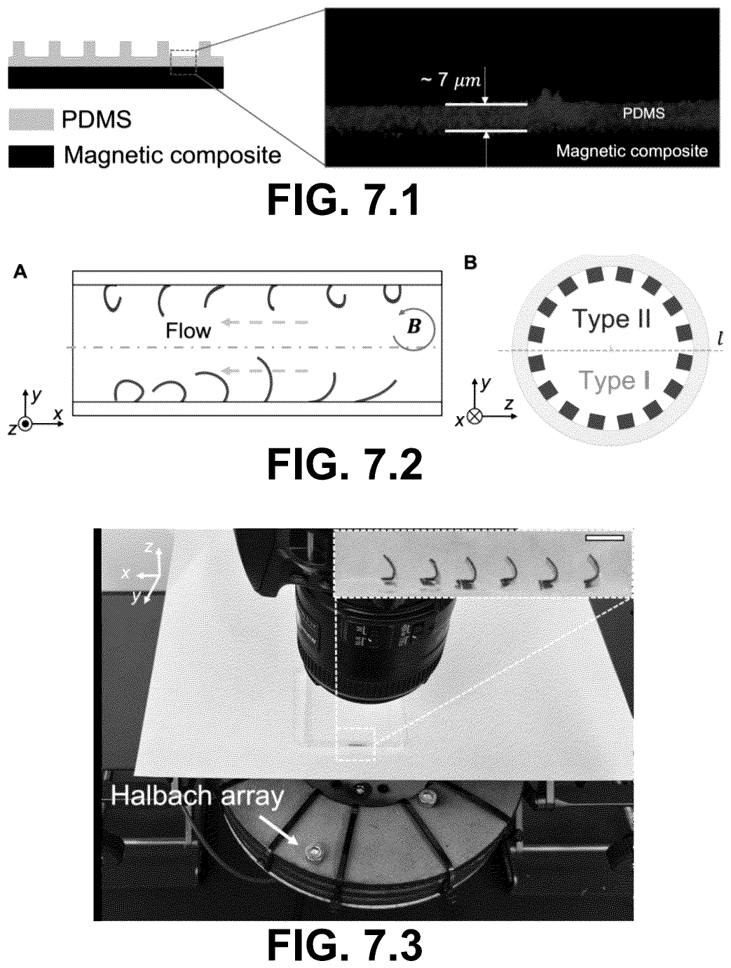

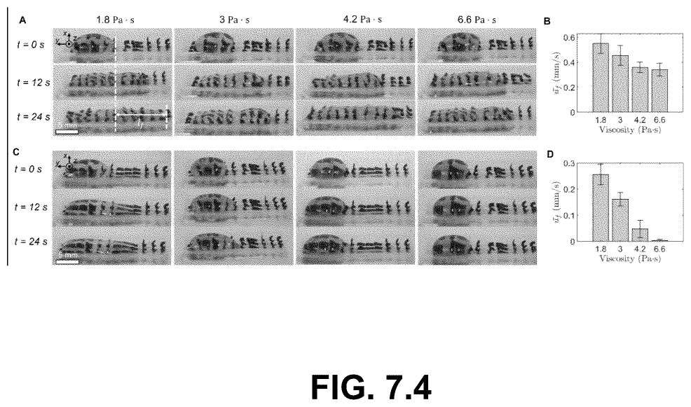

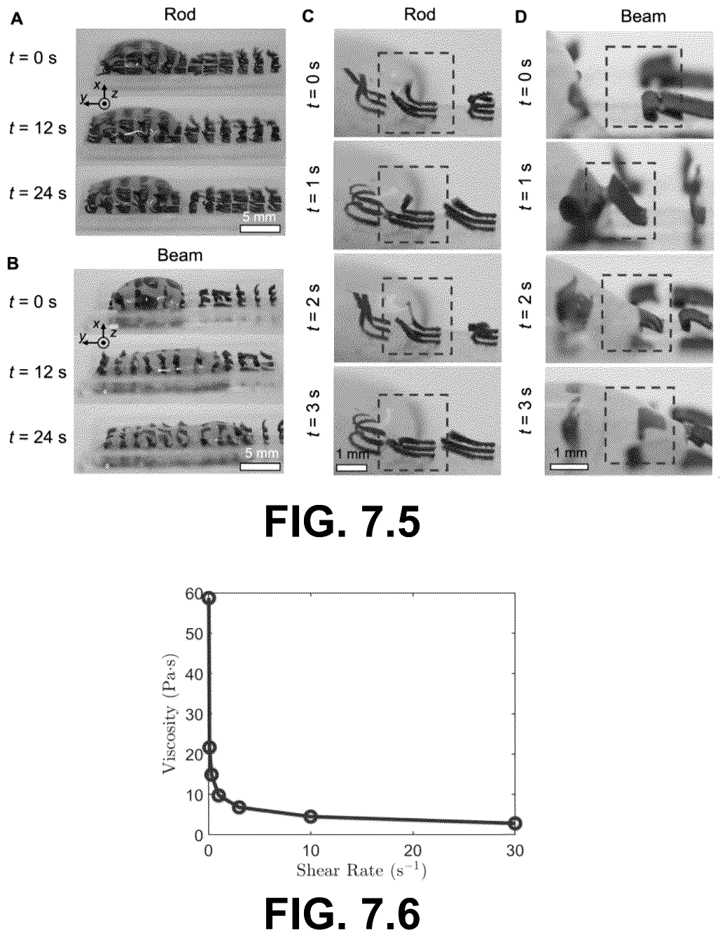

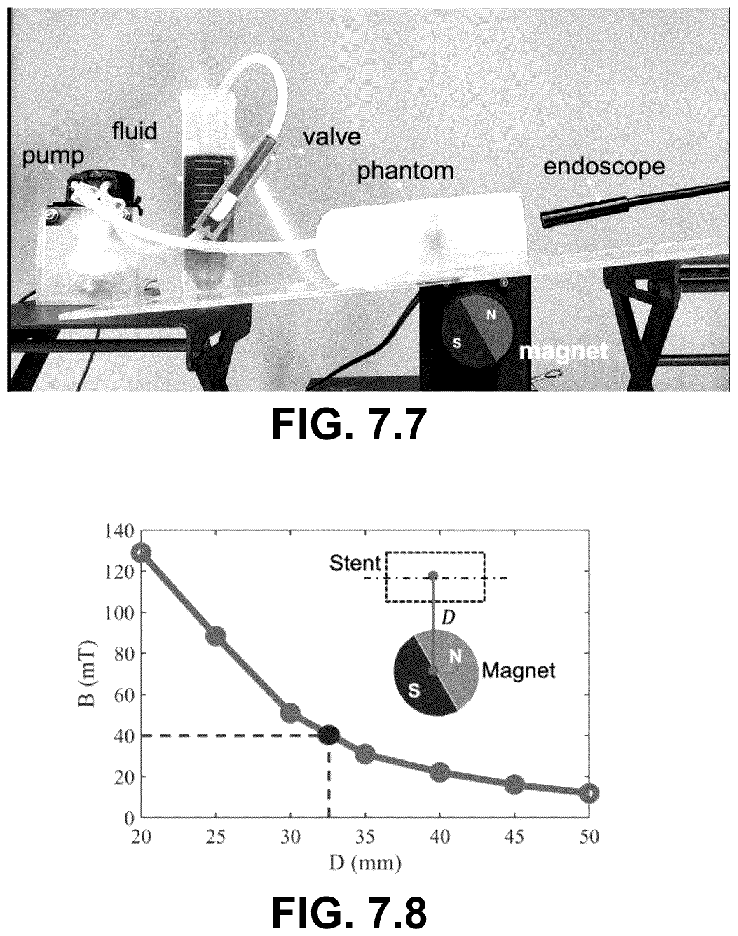

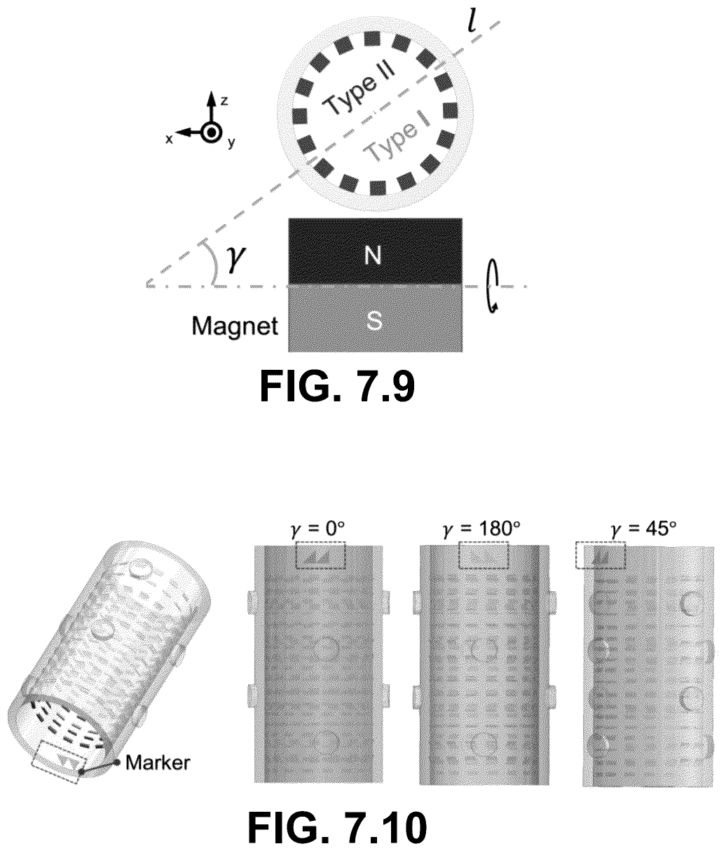

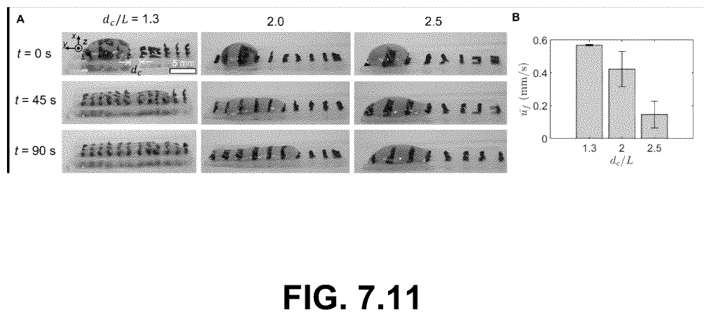

To manufacture the CAS, a fabrication method combining laser machining and micro-molding is proposed. FIG. 2(A) illustrates the overall fabrication process of a cilia patch (see “Fabrication of artificial cilia patches” in the “Materials and Methods” for more details), which will be bonded inside a hollow airway stent. First, the magnetic cilium has a three-layer structure including a magnetic composite layer made of NdFeB particles and silicone rubber (Ecoflex 0030, Smooth-On, Inc.) as the middle layer, and two microstructured PDMS layers on the top and bottom surfaces. The magnetic composite layer has a programmed magnetization profile to achieve a desired deformation when actuated by an external magnetic field. Second, as shown in FIG. 2(B), the PDMS layers encapsulate the magnetic composite layer tightly to improve the device biocompatibility. Third, patterning microstructures on the PDMS layers further increases the surface area to allow a robust hydrogel coating. The PDMS layers are fabricated using a spin-coating method to allow a thickness of about 7 μm (FIG. 7.1). Fourth, the fabricated cilia on a patch are further magnetized with a desired magnetization profile, when being placed inside an impulse magnetizer with a magnetic field of 2.2 T assisted with a magnetizing jig as shown in FIG. 2(C). For example, the two types of magnetization profiles provide cilia with either positive or negative SAs, such that a net flow in the same direction can be induced subject to a global rotating magnetic field (FIG. 7.2). The magnetizing jigs are designed to have a −π/3 phase delay in a cilium array to create an optimal metachronal wave for pumping fluids. Fifth, after being magnetized, the artificial cilia array is bonded to a soft back layer to form a cilia patch. To further coat the cilia patch with a thin layer of hydrogel for wetting-based pumping, a photo-initiator and PEGDA hydrogel monomer are applied sequentially on the cilia surface followed by UV exposure (FIG. 2(D)). The cilia patch will bend upon applying a magnetic field in the x-z plane (FIG. 2(E)). Lastly, the cilia patch is bonded to a stent back layer fabricated using a molding method (see “Fabrication of the silicone stent” in “Materials and Methods”) as shown in FIG. 2(F).

### Optimization of the Cilia Patches for Mechanical Transportation of Viscous Fluids

With the fabrication capability, the cilia patch design on a stent is optimized to maximize the transporting speed of viscous fluids. The design parameters are chosen as the length (L), thickness (t_{b}), and width (w) of a single cilium and the spacing do between two adjacent cilia bases as illustrated in FIG. 3(A). The spacing between the cilia arrays are denoted da which is fixed at 1.5 mm. The swiping area ratio rate (SARR) is defined as SA·f/(dₛ·d_{c}) is used as a metric to systematically quantify the fluid transportation ability per cilia patch area and per unit time, where f is the frequency of the actuation signal. A motorized array of permanent magnets called Halbach array is used to actuate the cilia patch as it can generate a uniformly distributed rotating magnetic field as shown in FIG. 3(B) and FIG. 7.3. The time-varying magnetic field at the center of the cilia patch is measured by a three-axis magnetic sensor (3D Magnetic sensor 2GO, Infineon Technologies) as shown in FIG. 3(C).

As a showcase of optimizing the cilia patch, we choose to investigate how the bending motion of the cilia affects the fluid pumping, by designing the beam length-to-thickness ratio and the magnetic field actuation signals. We fix the other cilium parameters with t_{b}=70 μm, dc=1.3 L and w=1.5 mm for ease of comparison. The magnetization profiles and kinematics of the two types of cilia (FIG. 2(C)) to be integrated in the stent are shown in FIG. 3(D). As shown in FIG. 3(E) and FIG. 3(F), both the length-to-thickness ratio (L/t′) and the actuation frequency f strongly affect the transportation speed for viscous fluids (glycerol). With a smaller L/t′, it is more difficult for the cilium to deform but easier to damp out due to fluid drag. The SSAR increases as f ramps from 1 Hz to 2 Hz but decreases after f is larger than 2 Hz. For Type I cilia, with t′=70 μm, the SSAR increases as the L increases from 1.5 mm to 1.75 mm but decreases as L further increases to 2 mm (FIG. 3(E)). Similarly in FIG. 3(F), the SSAR of Type II cilia increases as L increases from 1 mm to 1.25 mm but decreases as the length further increases to 1.5 mm. This is because initially by increasing the length, the area that the cilium swipes over during the power stroke gets larger, but it damps out earlier in the recovery stroke when the cilium length further increases.

To optimize the cilia motion when integrated on the curved stent surface, in FIG. 3(G), effect of the magnetic field's rotating plane on the cilia motion and pumping performance. The angle was defined between the magnetic field plane and the x-y plane as β. As β increases from 0 degrees, magnetic torques about the x- and y-axes will induce a twisting motion. As it is difficult to quantify the transportation performance by measuring the SSAR, the particle transportation speed was used to quantify the fluid transportation performance. FIG. 3(G) shows that the transportation speed of a particle (density: 1.2 g/cm³, diameter: 350 μm) decreases as the angle increases as the directional pumping is less effective due to the twisting motion. Nonetheless, the particle could still be transported with an average speed of 2.09 mm/s and 1.82 mm/s which are about 18% and 33% of the speeds when β=0 degree for Type I and Type II cilia, respectively. The magnetic field rotating plane can be adjusted to allow each cilia array on a curved stent surface to pump with the fastest speed within a specific duration such that each cilia array could still contribute to pumping fluids efficiently inside the stent. Finally, with the optimized cilia patch, the transportation speed of fluids of different viscosities are quantified in FIG. 3(H). Viscous liquids were prepared having several viscosities similar to that of airway mucus which is typically below 10 Pa·s. Within the cilia beating frequency is about 10 rad/s, FIG. 3(H) shows that the SARRs of the two types of cilia are 0.067 s⁻¹ and 0.029 s⁻¹, respectively in liquid with a viscosity of up to 20 Pa·s.

### Hydrogel Coating for Wetting-Based Transportation of Viscous Fluids

To further assist the mucus transportation, inspired by the human airway cilia that can maintain a proper hydration level of the mucus, the CAS was designed with hydrogel coating to increase their surface wettability for water. This is because hydrogel can trap water molecules to form a hydrophilic network. As water is one of the main components of mucus, a hydrogel-coated surface is hypothesized to allow mucus to spread out easily thanks to its strong hydrophilicity. To prove this effect, in FIG. 4, the wetting-based pumping of water-based viscous fluids by coating the cilia patches with hydrophobic and hydrophilic materials was investigated. As shown in FIG. 4(A), the cilia surface is coated with micro-structured PDMS for hydrophobic surface and then hydrogel on top of the PDMS surface for a hydrophilic surface. Compare with un-coated magnetic cilia surface (FIG. 4(A)(i)), the patch fully covered by a PDMS layer (FIG. 4(A)(ii)) is brighter due to an optical effect and is shown to have an average thickness of 7 μm visualized in a fluorescence microscope (FIG. 7.1). The hydrogel-coating layer (thickness: 20 μm) is visualized by a fluorescence microscope after adding Rhodamine 6G into the monomer solution as shown in FIG. 4(A)(iii). To verify the surface wetting properties, the water contact angle was measured for the artificial cilia with PDMS coating, and with hydrogel coating. FIG. 4(B) shows that with the micro-structured PDMS coating, the cilia surface is relatively hydrophobic with a water contact angle of 104 degrees. After further coating the cilia surface with hydrogel, the surface becomes hydrophilic with a water contact angle of 63 degrees. To investigate whether the hydrogel layer changes the bending stiffness of the cilia, the SARRs of the pure PDMS-coated and hydrogel-coated cilia are compared in FIG. 4(C). The hydrogel coated cilia has a slightly larger swiping area than the cilia without hydrogel coating. In this case, the hydrogel layer works as a lubricant layer which reduces the shear force on the cilia surface and enhances the non-reciprocal motion.

While a hydrophobic surface may reduce the adhesion between the water-based liquids and the cilia patch, it will pin the water-based liquids due to surface tension and cause liquid accumulation. The wetting effect was compared by adding a droplet of syrup onto a cilia patch with pure micro-structured PDMS coating (FIG. 4(D). A syrup droplet of 0.3 mL is spread to the other side in 30 seconds on a hydrogel-coated cilia patch, while a syrup droplet is pinned at its initial position, only deforming back and forth on a pure micro-structured PDMS coated cilia patch. To test the wetting-based pumping of mucus, in FIG. 4(E), 0.2 mL mucus was added on both types of cilia patches, the transportation speed is 2.5 times faster on the hydrogel coated surfaces compared with that on the one with pure micro-structured PDMS coating. This proves the effectiveness of a hydrogel-coated surface for enhanced mucus transportation.

### Integration, Control, and Characterization of the Ciliary Airway Stent

The optimized cilia patches are integrated in an airway stent and the effectiveness of the design is further validated for viscous liquid transportation. The CAS is embedded in a trachea phantom placed on a sample holder with an adjustable tilting angle α as shown in FIG. 5(A) and FIG. 7.4. A single spherical permanent magnet (diameter: 20 mm, NdFeB, N42) mounted on a stepper motor is placed under the sample holder at a distance of D to provide a rotating magnetic field of a controlled frequency up to 5 Hz and magnitude up to 40 mT by tuning D (FIG. 7.5). The average transportation speed when the liquid (syrup) is transported from one end of the stent to the other is quantified as illustrated in FIG. 5(B). The liquid is fed into the phantom at a relatively small speed (0.01 mL/s) by a peristaltic pump so that it could be at one end of the stent and further transported by the cilia to the other end. The average net transportation speed (u) by the cilia is further obtained with the overall liquid transportation speed subtracted by the liquid feeding speed.

To prove the effectiveness of the design, the pumping performance of five stents with different conditions is investigated in control experiments as shown in FIG. 5(C) and 5(D). Stent I and Stent III both have hydrogel-coated cilia, while Stent II and Stent IV only have cilia without hydrogel coating. The cilia inside Stent I and Stent II are actuated by the magnet, while they are held still in Stent III and Stent IV. Stent V is just a plain silicone stent without cilia for comparison. At t=100 s, Stent I with coating and active cilia transports the syrup with the longest distance, compared with other stents when syrup travels less than 50% of the distance using Stent I. The average transportation speeds are calculated and plotted in FIG. 5(D), which indicate a joint contribution of wetting-based pumping and motion-based pumping of viscous fluids. Stent I has a transportation speed of 0.17 mm/s (10.2 mm/min), which is twice of that of the mucociliary clearance speed in the human lung 5.5 mm/min and significantly higher than Stent II (0.07 mm/s, active cilia, no coating), Stent III (0.03 mm/s, passive cilia, coating), when there is only mechanical pumping or only wetting-based pumping. Since the transportation speeds are subtracted by the liquid feeding speed, they are zero for Stent IV and Stent V, which verifies that if there is not coating or active cilia motion, there is no transportation capability. With Stent I, its performance of pumping fluids of different viscosities is tested in FIG. 5(E). Liquids with different viscosities are also used to study Stent I's pumping performance. As the viscosity increases, the transportation speed decreases, but the stent can still pump fluids with a speed of 37 72.5 μm/s (4.4 mm/min) for liquid with a viscosity of 20 Pa·s, comparable with the mucociliary clearance speed in the human lung 5.5 mm/min. In addition, the ability of Stent I to pump viscous fluids against the gravity in were tested FIG. 5(F). A tilting angle α was produced between the phantom plane and the horizontal plane and find that when α=10 degrees, the stent can still transport the liquid though it is much slower than on a horizontal surface. It indicates that in the future the patients may not need to lie flat for the mucus clearance in clinical 1 applications. This angle may be further increased by optimizing the stent design. Lastly, as the stent has two types of magnetic cilia that are not symmetrically distributed about the body's axis, the effect of the stent orientation about the body's axis on the pumping performance is tested in FIG. 5(G). γ is defined as the angle between the division line (see FIG. 7.2) and the rotating axis of the magnet (see FIG. 7.6). When γ=0 or γ=180 degrees, the transportation speeds are similar for all cilia contacting the liquids as shown in FIG. 5(G). However, when γ=90 degrees, the transportation speed decreases suggesting that the stent should be implanted with γ=0 or γ=180 degrees.

### Transporting Mucus in a Lung Phantom and Visualization by Medical Imaging

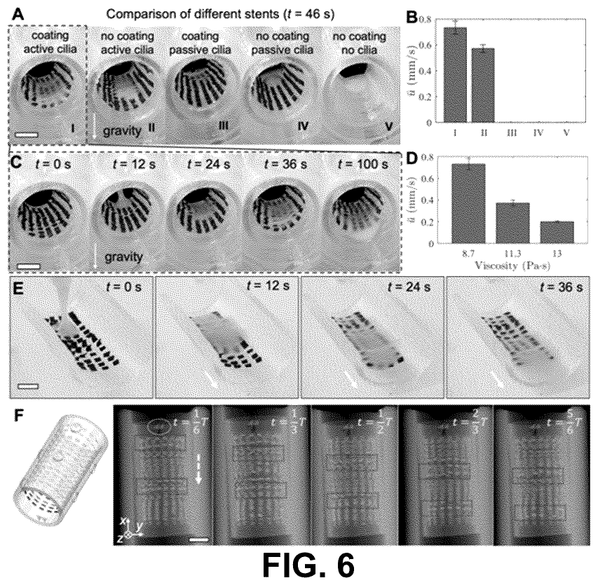

In FIG. 6, the performance of transporting mucus using the optimized CAS is tested. Mucus from the porcine stomach is prepared for the test (see “Viscous liquids” in “Materials and Methods” for the details). Mucus of a volume of 0.3 mL is first fed into different airway stents until they are stationary as shown in FIG. 6(A). Only stents with active cilia can transport the mucus to the other end indicating it is necessary to have the ciliary motion-based transportation mechanism for the mucus clearance in a stent. In addition, the hydrogel-coated ciliary stent (Stent I) shows a higher speed (0.7 mm/s) than the one coated with only PDMS (Stent II) as shown in FIG. 6(B), suggesting the wetting-based pumping enhances mucus transportation. Moreover, to show the pumping process more clearly, in FIG. 6(C) the video frames of the mucus transporting process in Stent I were presented. The mucus is initially fed from one end, settled down, and then pumped out of the cilia stent within 2 minutes. FIG. 6(D) shows that the stent can transport mucus of different viscosities from 8.5 Pa·s to 13 Pa·s. Finally, to visualize the pumping process together with the cilia motion inside the stent, FIG. 6(E) shows the process of pumping mucus in a “half” stent. The video frames show that 0.2 mL mucus is added and gradually transported, which gets thinner at the inlet side. The mucus eventually starts being pumped out of the stent after 36 seconds. Finally, the stent can be visualized using a medical imaging modality such as Computational Tomography (CT) for guiding the implantation in the lung trachea. The CAS was placed inside a lung trachea phantom made of elastomer with similar density to lung tissues, and the stent was visualized inside an X-ray medical imaging cabinet. To facilitate the precise implantation of the stent with a controlled orientation, the stent has two triangle-shaped markers to indicate the axial orientation and inlet-to-outlet orientation of the stent as shown in FIG. 6(F) and FIG. 7.7. FIG. 6(F) also shows that the ciliary motion, such as the metachronal wave, could be clearly visualized when applying a rotating magnetic field for potentially monitoring the status of the artificial cilia in vivo.

### DISCUSSION

A method to design and integrate magnetically actuated artificial cilia on 3D curved surfaces is presented by showcasing a ciliary airway stent for potential excessive mucus removal. The integrated magnetic artificial cilia exhibit metachronal coordination and non-reciprocal motions like their biological counterparts for efficient mechanical pumping of viscous fluids. The aspect ratio and magnetic actuation signals were particularly optimized for the artificial cilia to maximize the fluid transportation speed. Moreover, a wetting-based transportation mechanism has also been introduced and validated by coating hydrogel for transporting 1 water-based fluids. Finally, it has been demonstrated that the optimized cilia patches integrated on a silicone stent allow transportation of viscous synthetic fluids such as syrup, honey, and porcine mucus efficiently. With the mechanical pumping and the hydrogel-enabled wetting mechanism, the proposed CAS thus has shown unprecedented capability of removing excessive mucus in a minimally invasive manner. The CAS could be further improved towards potential therapeutic functions for patients at home. First, a layer of mucus of 1 mm in thickness tends to remain inside the stent, though they could trap dusts or bacteria and bring them back up to the oropharynx by the ciliary ‘elevator’ and coughed out. The thickness of the trapped mucus layer could be reduced by making the artificial cilia shorter with the same aspect ratio. Second, the maximum angle between the stent body axis and the horizontal plane could be further increased by enhancing the pumping ability so that patients wearing this device do not need to lie down during the procedure. In this way, the implantable stent with mucociliary clearance capability will potentially allow the patients to perform the therapy at home for a long-term. This may be realized by making shorter and more densely distributed cilia for a faster mucus pumping speed. Lastly, the magnetic actuation system could be further optimized for a better pumping performance and portability by developing portable magnetic actuation and control systems. In summary, the proposed ciliary airway stent overcomes the limitation of existing airway stents by reducing the frequency of bronchoscopic interventions needed for conventional silicone airway stents, avoiding the complications associated with tissue ingrowth with uncovered metal stents. The proposed method of integrating wirelessly actuated artificial cilia in airway stents is also potentially helpful for developing stents in other organs for pumping viscous fluids.

### Materials and Methods

Fabrication of artificial cilia patches. A Polyimide (PI) tape (thickness: 50 μm) was first bonded to a glass slide and subsequently patterned with an array of holes (diameter: 60 ym, spacing: 200 μm) by a laser cutter (LPKF U4, LPKF North American AG). The patterned PI tapes were made hydrophilic in a UV ozone machine (Model 18, Jelight Company, Inc) for 10 mins, and were further treated with 50 μL Trichloro(1H, 1H, 2H, 2H-perfluorooctyl) silane (Sigma-Aldrich, Co.) into a vacuum desiccator for 2 hours. Then, to coat artificial cilia, liquid PDMS (Dow Chemical Company, Sylgard silicone) with the elastomer base and curing agent according to a weight ratio of 20:1 was spin-coated onto the PI tape in a spin-coater (SCK-300P, Instras Scientific LLC) at 6000 rpm. After sitting on a hotplate at 90° C. for 10 mins, the PDMS was cured inside the cavities of the PI tapes. Two PI tapes with molded PDMS were further stacked together with a thin layer of magnetic composite in the middle, which was composed of Ecoflex-0030 (Smooth On, Inc.) and NdFeB (Magnequench International, LLC) with a weight ratio of 2:1. The thickness (70 μm) of the magnetic composite layer was controlled by tape spacers. After the magnetic composite was cured on a hotplate at 90° C. for 20 minutes, the glass slide and PI tape on one side was peeled off. The outlines of artificial cilia were then ablated on the three-layer structure using the laser cutter. Liquid PDMS was applied to the cut-out parts to coat the sides of the magnetic cilia which were exposed by the laser. During this process, a layer of PI tape was used to cover the top surface of micro-structured artificial cilia to prevent the structures from being coated by liquid PDMS. Extra liquid PDMS outside the cilia was carefully removed before the PDMS cured. Finally, the coated artificial cilia beams were bonded using Ecoflex-0031 (Smooth-On, Inc) on a thin layer of PDMS to form a cilia patch.
Hydrogel coating of an artificial cilia patch. The cilia were firstly deformed to point upward by putting a permanent magnet under it. Then they were submerged into Benzophenone-ethanol mixture (1:4 by weight) for 1 min. After that, the liquid was swiped clean with a tissue. PEGDA was then applied to cover the cilia patch surface with a pipette. Finally, UV lights shone on the cilia patch for 20 mins.
Fabrication of a silicone stent. The silicone stent with millimeter-pillar structures on the outside layer was fabricated by a sacrificial molding method. A negative mold with hollow structures was printed with water-soluble Polyvinyl alcohol (PVA) using a Fused Deposition Modeling (FDM) 3D printer. Liquid PDMS was injected into the negative mold, and subsequently cured on a hotplate at a temperature of 40° C. for 48 hours. After the PDMS was cured inside the mold, the whole structure was dissolved in hot water (70° C.) to obtain the stent.
Magnetic actuation. Two magnetic actuation systems were used. First, a Halbach array was used to actuate the artificial patches for optimizing the cilia structures (FIG. 7.3). The Halbach array was made of twelve identical NdFeB magnets of which each has a dimension of 25 mm by 25 mm by 25 mm. During the experiments, a cilia array was submerged inside a liquid and placed at 21 mm above the center of the Halbach array. The generated magnetic field was about 40 mT within a circular region with a radius of 10 mm (95% uniformity). A DC motor was further used for rotating the Halbach array to generate a rotating magnetic field. In addition, a spherical NdFeB magnet with a diameter of 32 mm was used to actuate the ciliary airway stent inside an airway phantom with a magnetic field up to 40 mT at 40 mm from its center. The 1 spherical magnet was further rotated by two step motors to provide a rotating magnetic field in different planes.
Experimental setup. A customized peristaltic pump (Gikfun Inc) actuated by a DC motor (FIG. 7.4) was used to feed testing liquids into the airway phantom. The liquid feeding speed was between 0.37 ml/min and 2.07 ml/min, which was controlled by a mechanical valve. The inlet of the feeding liquid was put through a hole on a plug inside the stent to block liquid flowing to the other direction due to gravity. A “Y”-shaped trachea phantom made of Ecoflex-0045 (Smooth On, Inc.) was fabricated using a sacrificial molding method similar to the one used for preparing the silicone stent. The obtained trachea phantom has a wall thickness of 8 mm and inner diameter of 20 mm. The shape of the trachea phantom and its stiffness could be adjusted by using different soft materials and different wall thickness.
Viscous liquids. The liquids of different viscosities were prepared by mixing water into different liquids including glycerol (Organic Verdana, μ=1200 mPa·s), syrup (Karo Light Corn Syrup, μ=5000 mPa·s), or honey (Private Selection, μ=20000 mPa·s) according to different weight ratios. For example, the syrup-water mixture of a weight ratio of 100:1, and the honey-water of a weight ratio of 100:1, have the viscosities of μ=2807 mPa·s and μ=10000 mPa·s, respectively. The viscosities of the liquid mixtures were measured by a viscometer (NDJ—8S, Bonvoisin). The mucus was prepared by mixing porcine mucin (Chem-impex International Inc.) with water according to different weight ratios. The mixtures were stirred for 1 hour at room temperature (20° C.). For example, with mucin-water mixtures with a ratio of 1:8, 1:7, and 1:6, have viscosities of μ=8705 mPa·s, μ=11307 mPa·s, and μ=13500 mPa·s at a shear rate of 1 s*+, respectively.

In addition to the foregoing, the various embodiments of the present disclosure include, but are not limited to, the embodiments set forth in the following clauses:
Clause 1—An apparatus, comprising a tubular member having a radially external side and a radially internal side; and a plurality of artificial cilia disposed circumferentially on the radially internal side of the tubular member, the plurality of artificial cilia forming arrays extending longitudinally along the radially internal side of the tubular member.
Clause 2—The apparatus of clause 1, further comprising a hydrogel coating on each of the plurality of artificial cilia.
Clause 3—The apparatus of clause 1 or 2, wherein the tubular member is a silicone airway stent.
Clause 4—The apparatus of any of clauses 1-3, wherein the plurality of artificial cilia are magnetically actuated to produce metachronal waves.
Clause 5—The apparatus of clause 4, wherein the plurality of artificial cilia further comprises a first plurality of artificial cilia disposed on a first semi-cylinder portion of the tubular member, the first plurality of artificial cilia having a first magnetization profile; and a second plurality of artificial cilia disposed on a second semi-cylinder portion of the tubular member, the second plurality of artificial cilia having a second magnetization profile.
Clause 6—The apparatus of clause 5, wherein the first magnetization profile and the second magnetization profile are configured such that a magnetic actuator can cause both the first plurality of artificial cilia and the second plurality of artificial cilia to produce a net flow from an entry end to an exit end of the tubular member.
Clause 7—The apparatus of any of clauses 4-6, wherein individual artificial cilia of the plurality of artificial cilia further comprises a first layer of microstructured Polydimethylsiloxane (PDMS); a magnetic composite layer disposed atop the first layer of PDMS, the magnetic composite layer having a programmed magnetization profile to achieve metachronal wave-like deformation when actuated by an external magnetic field; and a second layer of microstructured PDMS disposed atop the magnetic composite layer.
Clause 8—A method of manufacturing a ciliary airway stent, comprising forming a three-layer structure from Polydimethylsiloxane (PDMS) and a magnetic composite; laser cutting a plurality of artificial cilia from the three-layer structure; bonding the plurality of artificial cilia on a layer of PDMS to form an artificial cilia patch; and bonding the artificial cilia patch inside a hollow tubular member.
Clause 9—The method of clause 8, further comprising applying liquid PDMS to exposed sides of the plurality of artificial cilia after laser cutting; and curing the liquid PDMS.
Clause 10—The method of clause 8 or 9, further comprising applying a hydrogel coating to the plurality of artificial cilia.
Clause 11—The method of clause 10, wherein applying the hydrogel coating further comprises submerging the artificial cilia patch in benzophenone-ethanol; drying the artificial cilia patch; applying polyethylene glycol diacrylate (PEGDA) to the artificial cilia patch; and curing the PEGDA with ultra-violet light.
Clause 12—The method of any of clauses 8-11, wherein forming the three-layer structure further comprises molding a first layer of PDMS to have an array of recesses; layering a magnetic composite over the first layer of PDMS; layering a second layer of PDMS over the magnetic composite; and curing the first layer and the second layer of PDMS together over the magnetic composite.
Clause 13—The method of clause 12, wherein laser cutting the plurality of artificial cilia from the three-layer structure is based at least in part on the array of recesses molded into the first layer of PDMS.
Clause 14—The method of any of clauses 8-13, further comprising injecting liquid PDMS into a negative mold; and curing the PDMS to form the hollow tubular member.
Clause 15—A system, comprising a tubular member having a radially external side and a radially internal side; a plurality of magnetic cilia disposed circumferentially on the radially internal side of the tubular member, the plurality of magnetic cilia extending along the radially internal side of the tubular member from an entry end to an exit end; and a magnetic actuation system configured to generate metachronal wavelike motion of the plurality of magnetic cilia.
Clause 16—The system of clause 15, wherein the plurality of magnetic cilia further comprises a first plurality of magnetic cilia disposed on a first semi-cylinder portion of the tubular member, the first plurality of magnetic cilia having a first magnetization profile; and a second plurality of magnetic cilia disposed on a second semi-cylinder portion of the tubular member, the second plurality of magnetic cilia having a second magnetization profile.
Clause 17—The system of clause 16, wherein the first magnetization profile and the second magnetization profile are configured such that the magnetic actuation system can cause both the first plurality of magnetic cilia and the second plurality of magnetic cilia to produce a net flow from the entry end to the exit end of the tubular member.
Clause 18—The system of any of clauses 15-17, further comprising a hydrogel coating on each of the plurality of magnetic cilia.
Clause 19—The system of any of clauses 15-18, wherein the magnetic actuation system comprises a rotating Halbach array of a plurality of magnets.
Clause 20—The system of any of clauses 15-19, wherein individual magnetic cilia of the plurality of magnetic cilia further comprises a first layer of microstructured polydimethylsiloxane (PDMS); a magnetic composite layer disposed atop the first layer of PDMS, the magnetic composite layer having a programmed magnetization profile to achieve metachronal wave-like deformation when actuated by an external magnetic field; and a second layer of microstructured PDMS disposed atop the magnetic composite layer.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. An apparatus, comprising: a tubular member having a radially external side and a radially internal side; and
a plurality of artificial cilia disposed circumferentially on the radially internal side of the tubular member, the plurality of artificial cilia forming arrays extending longitudinally along the radially internal side of the tubular member.

2. The apparatus of claim 1, further comprising a hydrogel coating on each of the plurality of artificial cilia.

3. The apparatus of claim 1, wherein the tubular member is a silicone airway stent.

4. The apparatus of claim 1, wherein the plurality of artificial cilia are magnetically actuated to produce metachronal waves.

5. The apparatus of claim 4, wherein the plurality of artificial cilia further comprises:
a first plurality of artificial cilia disposed on a first semi-cylinder portion of the tubular member, the first plurality of artificial cilia having a first magnetization profile; and
a second plurality of artificial cilia disposed on a second semi-cylinder portion of the tubular member, the second plurality of artificial cilia having a second magnetization profile.

6. The apparatus of claim 5, wherein the first magnetization profile and the second magnetization profile are configured such that a magnetic actuator can cause both the first plurality of artificial cilia and the second plurality of artificial cilia to produce a net flow from an entry end to an exit end of the tubular member.

7. The apparatus of claim 4, wherein individual artificial cilia of the plurality of artificial cilia further comprises:
a first layer of microstructured Polydimethylsiloxane (PDMS);
a magnetic composite layer disposed atop the first layer of PDMS, the magnetic composite layer having a programmed magnetization profile to achieve metachronal wave-like deformation when actuated by an external magnetic field; and
a second layer of microstructured PDMS disposed atop the magnetic composite layer.

8. A method of manufacturing a ciliary airway stent, comprising:
forming a three-layer structure from Polydimethylsiloxane (PDMS) and a magnetic composite;
laser cutting a plurality of artificial cilia from the three-layer structure;
bonding the plurality of artificial cilia on a layer of PDMS to form an artificial cilia patch; and
bonding the artificial cilia patch inside a hollow tubular member.

9. The method of claim 8, further comprising:
applying liquid PDMS to exposed sides of the plurality of artificial cilia after laser cutting; and
curing the liquid PDMS.

10. The method of claim 8, further comprising applying a hydrogel coating to the plurality of artificial cilia.

11. The method of claim 10, wherein applying the hydrogel coating further comprises:
submerging the artificial cilia patch in benzophenone-ethanol;
drying the artificial cilia patch;
applying polyethylene glycol diacrylate (PEGDA) to the artificial cilia patch; and
curing the PEGDA with ultra-violet light.

12. The method of claim 8, wherein forming the three-layer structure further comprises:
molding a first layer of PDMS to have an array of recesses;
layering a magnetic composite over the first layer of PDMS;
layering a second layer of PDMS over the magnetic composite; and
curing the first layer and the second layer of PDMS together over the magnetic composite.

13. The method of claim 12, wherein laser cutting the plurality of artificial cilia from the three-layer structure is based at least in part on the array of recesses molded into the first layer of PDMS.

14. The method of claim 8, further comprising:
injecting liquid PDMS into a negative mold; and
curing the PDMS to form the hollow tubular member.

15. A system, comprising:
a tubular member having a radially external side and a radially internal side;
a plurality of magnetic cilia disposed circumferentially on the radially internal side of the tubular member, the plurality of magnetic cilia extending along the radially internal side of the tubular member from an entry end to an exit end; and
a magnetic actuation system configured to generate metachronal wavelike motion of the plurality of magnetic cilia.

16. The system of claim 15, wherein the plurality of magnetic cilia further comprises:
a first plurality of magnetic cilia disposed on a first semi-cylinder portion of the tubular member, the first plurality of magnetic cilia having a first magnetization profile; and
a second plurality of magnetic cilia disposed on a second semi-cylinder portion of the tubular member, the second plurality of magnetic cilia having a second magnetization profile.

17. The system of claim 16, wherein the first magnetization profile and the second magnetization profile are configured such that the magnetic actuation system can cause both the first plurality of magnetic cilia and the second plurality of magnetic cilia to produce a net flow from the entry end to the exit end of the tubular member.

18. The system of claim 15, further comprising a hydrogel coating on each of the plurality of magnetic cilia.

19. The system of claim 15, wherein the magnetic actuation system comprises a rotating Halbach array of a plurality of magnets.

20. The system of claim 15, wherein individual magnetic cilia of the plurality of magnetic cilia further comprises:
a first layer of microstructured polydimethylsiloxane (PDMS);
a magnetic composite layer disposed atop the first layer of PDMS, the magnetic composite layer having a programmed magnetization profile to achieve metachronal wave-like deformation when actuated by an external magnetic field; and
a second layer of microstructured PDMS disposed atop the magnetic composite layer.
